(19) United States
(12) Patent Application Publication
(10) Pub. No.: US 2010/0112035 A1
(43) Pub. Date: May 6, 2010

(54) MULTIPLE COMPONENT FOOD PRODUCT USEFUL FOR DELIVERING GLUCOSAMINE AND/OR N-ACETYL-D-GLUCOSAMINE

(75) Inventors: Brent Daniel ROGERS, Ottumwa, IA (US); Lawrence E. Fosdick, Oskaloosa, IA (US); John A. Bohlmann, Ottumwa, IA (US); Paula A. DeGrandis, Plymouth, MN (US)

Correspondence Address: CARGILL, INCORPORATED, P.O. Box 5624, MINNEAPOLIS, MN, 55440-5624, US

(73) Assignee: CARGILL, INCORPORATED, Wayzata, MN (US)

(21) Appl. No.: 12/613,386

(22) Filed: Nov. 5, 2009

Related U.S. Application Data
(63) Continuation of application No. 10/533,477, filed on Apr. 29, 2005, now abandoned.
Application No. 10/533,477 is a continuation-in-part of application No. 10/685,125, filed on Oct. 13, 2003.
Application No. 10/685,125 is a continuation-in-part of application No. 10/326,549, filed on Dec. 19, 2002, now Pat. No. 7,049,433.
Application No. 10/326,549 is a continuation of application No. 09/785,695, filed on Feb. 16, 2001, now abandoned.
Application No. PCT/US03/34668 is a continuation-in-part of application No. 10/326,549, filed on Dec. 19, 2002, now Pat. No. 7,049,433.
(60) Provisional application No. 60/423,119, filed on Nov. 1, 2002.

(51) Int. Cl. A61K 9/00; A61K 31/70; G01N 33/02

(52) U.S. Cl. 424/439; 514/62; 436/20

(57) ABSTRACT

Food products containing at least two components are provided. These products have at least one baked component and at least one non-baked component. The non-baked component contains either GLCN and/or NAG.

### CROSS REFERENCE TO RELATED APPLICATIONS

This is a continuation of U.S. application Ser. No. 10/533,477, filed Apr. 29, 2005 which is the US National Stage of International Application No. PCT/US2003/034668, filed Oct. 31, 2003 (published in English under PCT Article 21(2)), which in turn claims the benefit of U.S. Provisional Application No. 60/423,119, filed Nov. 1, 2002, and is a continuation-in-part of U.S. patent application Ser. No. 10/685,125 filed Oct. 13, 2003, which is a continuation-in-part of copending U.S. patent application Ser. No. 10/326,549 filed Dec. 19, 2002, which is a continuation of U.S. patent application Ser. No. 09/785,695 filed Feb. 16, 2001, and which claims priority from PCT Application No. PCT/US02/04468 filed Feb. 15, 2002, each of which is incorporated herein by reference.

### FIELD

This application relates to food products, and methods for making food products, that contain at least two components, a baked phase and a non-baked phase. The non-baked phase is characterized as containing glucosamine (GLCN) and/or N-acetyl-D-glucosamine (NAG).

### BACKGROUND

Dietary supplements for cartilage health are effective in reducing the symptoms of osteoarthritis and joint pain. Examples of such cartilage health supplements include glucosamine (GLCN) hydrochloride, GLCN sulfate, N-acetyl-glucosamine, chondroitin sulfate, hyaluronic acid (which is comprised of a repeating disaccharide of N-acetyl-D-glucosamine and D-glucuronic acid), and cetyl myristoleate (CM). The most commonly used cartilage health supplements are GLCN either in a hydrochloride or sulfate form.

It has previously been noted that GLCN is not stable at high temperatures, see U.S. Pat. No. 6,432,929. Therefore, in an attempt to avoid degradation of the cartilage health supplements U.S. Pat. No. 6,432,929 teaches that beverages that include GLCN are prepared using a process that requires two separate heating steps, to minimize chemical alteration of GLCN. The juice drink base is prepared using pasteurization at about 195° F. for 42 seconds. A separate GLCN water-based solution is prepared at a temperature of below 160° F. These two solutions are mixed to form the GLCN-supplemented beverage. U.S. Pat. No. 6,432,929 also teaches incorporating cartilage health supplements into snack bars that are not heat-treated.

Therefore, a need exists to develop a broader range of food products that can be used to deliver cartilage health supplements.

### SUMMARY

Surprisingly, and contrary to the teaching of the art, it has been found that some heat treatment conditions do not significantly degrade GLCN and that in some embodiments adjustments to the pH of products incorporating GLCN and/or NAG do not need to be made. However, conditions presented in baking processes do significantly alter GLCN concentration, and to a much lesser extent NAG concentration. Accordingly, disclosed herein are products and methods of making such products that contain at least one baked component and at least one non-baked component, wherein GLCN and/or NAG is contained in a non-baked component.

More specifically, the data provided herein show that as much as 87% of the available GLCN is lost after GLCN is subjected to baking and as much as 23% of available NAG is lost after baking (see Example 7, below). Hence, it can be difficult to accurately predict the amount of GLCN and/or NAG remaining in a baked product. Particular embodiments of the products and methods described herein, however, enable food formulators to make good tasting products that deliver a known amount of GLCN and/or NAG to the consumer.

### DETAILED DESCRIPTION OF SEVERAL EMBODIMENTS

As used herein and in the appended claims, the singular forms “a” or “an” or “the” include plural references unless the context clearly dictates otherwise. For example, reference to “a cartilage supplement” includes a plurality of such cartilage supplements and reference to “the food product” includes reference to one or more food products and equivalents thereof known to those skilled in the art, and so forth. Similarly, the word “or” is intended to include “and” unless the context clearly indicates otherwise. Hence “comprising A or B” means including A, or B, or A and B.

Unless explained otherwise, all technical and scientific terms used herein have the same meaning as commonly understood to one of ordinary skill in the art to which this disclosure belongs.

Unless otherwise indicated, all numbers expressing quantities of ingredients, properties such as molecular weight, percentages, reaction conditions, and so forth used in the specification and claims are to be understood as being modified by the term “about.” Accordingly, unless indicated to the contrary, the numerical parameters set forth are approximations that may depend upon the desired properties sought.

### Making a Baked Component

As used herein, the term “baking” includes processes such as toasting, that involve applying effective energy in the form of heat, utilizing an oven or suitable heating transfer apparatus capable of providing and directing heat to a dough (a soft flour mixture which is typically an emulsion of starches, proteins, sugars, water, fats and leavening agents) to convert a dough to a baked product (a product that has become firmer than when it is in the dough form). Baked items can contain flours such as soy, wheat, rice or corn. In some embodiments, the dough can also include GLCN and/or NAG, however, in some examples the final concentration of GLCN and/or NAG in the baked component will be less than in the same component prior to baking.

As used herein, the term “heat” includes convection, radiation, and/or microwave energy applied in a form effective and sufficient to accomplish baking of a product. “Baking” may involve exposing a product to a combination of one or more types of heat, e.g., convection, radiation and/or microwave energy. For example, a product may be “baked” by exposure to heat via an oven or convection and radiation. Baking mechanisms including electric, gas, wood and solar stoves, ovens, and furnaces can be employed if desired to achieve a desired temperature level. An open fire or hearth can be employed as a baking mechanism. The amount of time employed in cooking is determined from a number of factors including the amount of dough and composition to be baked, the water content and other variables of the baking operation.

Typically baking as described herein, refers to exposing dough to temperatures of at least about 200° F., at least about 250° F., at least about 300° F., at least about 350° F., or at least about 400° F. As mentioned above, the length of exposure varies on the composition of the dough being baked, and the amount of dough being baked.

Various illustrative useful types of ovens and suitable heating and baking apparatus, kneaders, mixing equipment and other apparatus useful in preparing and baking a dough based product to form a dough and to form a baked product from dough are disclosed in Perry's Chemical Engineers' Handbook, Seventh Edition, Robert H. Perry and Don W. Green, ISBNO 049841-5, 1997.

### Making a Non-Baked Component

The non-baked component of the food products described herein, includes fillings such a pie fillings, fillings such as those found in sandwich cookies such as OREO® and Nutter Butter® cookies, Twinkies®, doughnuts (jelly and cream), frostings and coatings, such as chocolate coatings on doughnuts or cereal bars, and sweetener coatings on cereals, gelatin, frosting, agglomerating material, such as marshmallow mix in Rice Krispies® treats, etc. Non-baked products are typically not dough based and they can be at any pH, such as a pH of at least 5, 6, 7, 8, or 9. At least one of the non-baked components of the food product includes a cartilage health supplement such as GLCN and/or NAG. NAG as used herein, refers to monomers of NAG, as well as oligomers of NAG, which have the same or similar thermal tolerance as disclosed herein. For example, NAG and NAG oligomers can be introduced into a food, and be subsequently subjected or exposed to a high temperature, without a resulting significant adverse effect on the taste, color, odor, and texture of the food product supplemented with NAG.

Oligomers of NAG are those that have a degree of polymerization, such as a polymer of 2-6 NAG molecules. Examples of NAG oligomers include, but are not limited to: dimers, trimers, tetramers, pentamers, and hexamers of NAG, which have the same or similar thermal tolerance as disclosed herein.

Depending upon the embodiment of the disclosed food product, NAG and GLCN can be obtained from particular suitable sources. In particular examples NAG or GLCN is derived from shellfish, fungal biomass, bacteria, and/or cartilage. In one example, NAG or GLCN is derived from fungal biomass containing chitin (for example see PCT Publication WO 03/013435).

During processing the non-baked component of the disclosed food products can be exposed briefly to high temperatures (heat processed), however, those temperatures will not substantially alter the concentration of GLCN and/or NAG in the non-baked component. More specifically, the non-baked component can be treated with heat as long as it maintains at least about 50%, at least about 60%, at least about 70%, at least about 80%, at least about 85%, at least about 90%, or at least about 95% of the pre-high temperature exposure concentration. That is, the concentration before temperature exposure will be substantially the same at the concentration in the finished product, thus, allowing for accurate labeling of the concentration in the finished product. The amount of GLCN and/or NAG added to a food product will depend on the desired concentration in the final product. The following non-limiting examples include about 0.001 g cartilage supplement per serving, such as at least 0.01 g/serving, such as at least about 0.05 g/serving, at least about 0.1 g/serving, at least about 0.25 g/serving, at least about 0.5 g/serving, at least about 0.75 g/serving, at least about 1.0 g/serving, at least about 1.5 g/serving, at least about 3.0 g/serving, at least about 5.0 g/serving, at least about 10.0 g/serving, or even at least about 20.0 g/serving. Moreover, any ratio of GLCN to NAG can be used to make a product at a given concentration, however, the disclosure is particularly useful in applications where the majority, or all of the cartilage health supplement is GLCN.

The GLCN and/or NAG-supplemented food products disclosed herein can be further supplemented with one or more other cartilage health supplements, vitamins, minerals, fats, proteins, sweeteners, organic acids, carbohydrates, or combinations thereof. In addition, other agents that treat cartilage dysfunction or skin disorders can also be included in the disclosed GLCN and/or NAG supplemented food products.

“High temperature” as used herein, refers to temperatures typically used when non-baked components are processed thermally or by irradiation. High temperatures can be achieved using by a combination of one or more of wood stoves, convection, radiation and/or microwave energy application means such as an oven or convection and radiation application means. Particular, non-limiting examples of high temperature include, temperatures of at least about 160° F., such as temperatures of at least about 180° F., at least about 200° F., at least about 240° F., at least about 300° F., at least about 325° F., at least about 350° F., at least n about 375° F., at least about 400° F., at least about 450° F., and even at least about 500° F. Particular, non-limiting examples of high temperatures used in pasteurization include, temperatures in the range of about 160° F.-about 280° F., such as about 195° F. for about 42 seconds (such as about 195±4° F. for about 42±4 seconds), about 200° F. for less than 40 seconds (such as about 200±5° F. for about 40±5 seconds), about 165° F. for about 3 minutes (such as about 165±5° F. for about 180±10 seconds).

### Methods of Making Food Products

The food products described herein can be made by any method known in the art for contacting non-baked components of food products with baked components of food products. Such methods include, but are not limited to, layering the components, incorporating (mixing) the components, spraying one component onto the non-baked component (see U.S. Pat. No. 4,079,151), enrobing one component with another component (see U.S. Pat. No. 5,876,775), injecting one component into the other component, and combinations thereof.

The finished food products (i.e., the combined non-baked component and the baked component) will typically be created such that they contain a target concentration of cartilage health supplement on a per serving basis. A serving is the amount of food a person or animal would customarily eat at one time. The serving size can often times be found on the Nutrition Facts label on the food product. Serving sizes are also shown on the USDA Food Pyramid. For bulk products, such as breakfast cereal and flour, a serving is usually represented in common household terms, such as cup, tablespoon, teaspoon, or fluid ounce. For products that come in discrete units, such as bread and cookies, a serving size is usually listed as the number of units that constitute a serving, such as three cookies or two slices of bread.

The disclosure provided herein allows for the generation of products that accurately deliver a therapeutically effective amount of cartilage health supplement. A therapeutically effective amount is an amount sufficient to achieve a desired biological effect, whether by itself or combined with other cartilage health supplement products to achieve a desired biological effect. In one example, it is an amount that is effective, whether by itself or combined with other cartilage health supplement products, to alleviate or reduce symptoms associated with cartilage dysfunction, such as pain, swelling, and/or decreased mobility, by more than a desired amount. In another example, it is an amount that is effective, whether by itself or combined with other cartilage health supplement products, to stabilize symptoms associated with cartilage dysfunction, such that the symptoms do not worsen. In particular examples, it is a concentration of GLCN and/or NAG that is effective, whether by itself or combined with other cartilage health supplement products, to alleviate, reduce, and/or stabilize symptoms associated with cartilage dysfunction, such as in a subject to whom GLCN and/or NAG is administered.

In one example, it is an amount that is effective, whether by itself or combined with other cartilage health supplement products, to alleviate or reduce symptoms associated with a skin disorder, such as promoting the healing of a wound or reducing the appearance of wrinkles, by more than a desired amount. In another example, it is an amount that is effective, whether by itself or combined with other cartilage health supplement products, to stabilize symptoms associated with a skin disorder, such that the symptoms do not worsen. In particular examples, it is a concentration of GLCN or NAG that is effective, whether by itself or combined with other cartilage health supplement products, to alleviate, reduce, and/or stabilize symptoms associated with a skin disorder, such as in a subject to whom GLCN or NAG is administered.

In one example, a therapeutically effective amount also includes a quantity of GLCN and/or NAG sufficient to achieve a desired effect, whether alone or combined with other cartilage health supplement products, in a subject being treated. For instance, it can be an amount necessary, whether alone or combined with other cartilage health supplement products, to improve signs and/or symptoms a disease, such as osteoarthritis, a skin disorder, or a wound.

The GLCN and/or NAG-containing food products disclosed herein have equal application in medical and veterinary settings. Therefore, the general term “subject being treated” is understood to include all animals (such as humans, apes, dogs, cats, horses, and cows) that require treatment of a cartilage dysfunction or skin disorder.

NAG has a higher thermal tolerance compared to GLCN, which refers to the ability of NAG to be exposed to a high temperature, without a resulting significant adverse effect on the taste, color, odor, and/or texture of a food supplemented with NAG, when NAG is present in the food during exposure to a high temperature. In contrast, GLCN is not as thermally tolerant, because when GLCN is exposed to high temperatures, the resulting food product often times has one or more undesirable characteristics, such as an unpleasant taste or undesirable browning, when GLCN is present in the food during exposure to a high temperature.

Using GLCN and/or NAG to Enhance Health

A method of treating a cartilage dysfunction in a subject by administering the disclosed GLCN and/or NAG-supplemented food products, is disclosed. In some examples, treatment alleviates or reduces the symptoms of cartilage dysfunction, such as increases joint mobility, reduces pain and/or reduces swelling in the subject. In some examples, treatment stabilizes the symptoms of cartilage dysfunction, such that the cartilage dysfunction is not exacerbated. Examples of cartilage dysfunction include, but are not limited to, joint pain and osteoarthritis.

Also disclosed are methods of treating a skin disorder in a subject by providing the disclosed GLCN and/or NAG-supplemented food products to a subject. In some examples, ingestion alleviates or reduces the symptoms of a skin disorder, such as promotes wound healing in the subject. In some examples, taking GLCN and/or NAG stabilizes the symptoms of a skin disorder, such that the skin disorder is not exacerbated. Examples of skin disorders include, but are not limited to, wounds and wrinkles.

A method for treating food allergies in a subject by administering the disclosed GLCN and/or NAG-supplemented food products to the subject is disclosed. In some examples, treatment alleviates or reduces the symptoms of a food allergy, such as reduces the inflammatory response to the food in the subject. In some examples, treatment stabilizes the symptoms of a food allergy, such that the food allergy is not exacerbated.

The subject treated can be a human or veterinary subject suffering from cartilage dysfunction, skin disorder or food allergy (for example see WO 93/14766A1). An effective amount of GLCN and/or NAG can be administered in a single serving, or in several servings, for example daily, during a course of treatment. However, the effective amount can depend on the subject being treated, the severity and type of the condition being treated, and the manner of administration. A typical amount of GLCN and/or NAG delivered in dietary supplement products is about 1.5 g/day, in a single or in multiple administrations. For example, if the subject was to receive multiple administrations in a single day, the subject might receive three servings of GLCN and/or NAG, each serving containing about 0.5 g NAG. NAG can administered at about at least about 0.001 g NAG/day. In one example, NAG is administered at about 0.75 g/day. In other examples, GLCN and/or NAG is administered at about at least 0.01 g/day, such as about at least 0.05 g/day, about at least 0.1 g/day, about at least 0.25 g/day, about at least 0.5 g/day, about at least 0.75 g/day, about at least 1.0 g/day, about at least 1.5 g/day, about at least 3.0 g/day, about at least 5.0 g/day, about at least 10.0 g/day, or even about at least 20.0 g/day.

GLCN from Fungal Biomass

Embodiments of the food products disclosed herein may use fungal biomass derived glucosamine compositions. The glucosamine compositions derived from fungal useful for forming embodiments of the disclosed food products may include particular components in addition to glucosamine, such as glucose, unreacted chitin, and glucan conversion materials, such as melanoidins and levulinic acid.

Melanoidins are relatively complex, high molecular weight, irregular polymers and are present in particular embodiments of the glucosamine compositions. For example, particular embodiments of the disclosed glucosamine compositions include from 0.001 to 15 wt. % melanoidins, or from 0.001 to 1.0 wt. % melanoidins or from 0.01 to 0.1 wt. % melanoidins. Without being tied to any particular theory, melanoidins are likely formed by the conversion of glucans to dextrose to hydroxymethylurfural (HMF) to produce the melanoidins. (The reaction may produce other glucan-derived products and amines from proteins in a biomass source as well as lipids in such a source.) Such a chemical process is known as the Maillard Reaction.

Levulinic acid (also known as acetyl-propionic acid) is present in particular embodiments of the disclosed glucosamine compositions. Without being tied to any particular theory, levulinic acid is likely formed when glucans in the fungal biomass are converted to dextrose, which is converted to HMF to finally form formic and levulinic acids. Levulinic acid is a non-hazardous component that is a valuable acidulant used in such products as carbonated and fruit juice beverages, jams, and jellies. Thus, addition of embodiments of the glucosamine compositions to such products provides an acidulant benefit as well as the benefits provided by the glucosamine in the composition. Particular embodiments of the disclosed food products include fungal biomass-derived glucosamine compositions having from 0.0001 to 1 wt. % levulinic acid, or from 0.001 to 0.7 wt. % levulinic acid or from 0.01 to 0.4 wt. % levulinic acid.

Because the melanoidins and levulinic acid are formed when producing the glucosamine compositions according to aggressive acid hydrolysis methods, no additional steps must be taken to include such components in the compositions. Melanoidins and levulinic acid are not present in glucosamine compositions derived from shellfish.

With reference to Table 1, embodiments of the glucosamine compositions useful for making embodiments of the presently disclosed food products comprise glucosamine derived from fungal biomass and may also comprise one or more of the listed components in Table 1, those shown in Table 2 and other components as discussed herein. Concentrations of each component may be within the ranges shown or may be varied by altering any of a variety of production parameters.

TABLE 1
Components that can be present in a GLCN food product.

| Glucosamine | Representative | Representative | Representative |
|---|---|---|---|
| Composition | Embodiment | Embodiment | Embodiment |
| Components | Percent by Weight | Percent by Weight | Percent by Weight |
| Glucosamine | 85-99.8 | 95-99.8 | 98-99.8 |
| Melanoidins | 0.001-15 | 0.001-1.0 | 0.01-0.1 |
| Levulinic Acid | 0.0001-1 | 0.001-0.7 | 0.01-0.4 |
| Dextrose | 0.001-10 | 0.001-5 | 0.001-2 |
| Citric Acid | 0.001-10 | 0.01-1.0 | 0.025-0.5 |

With reference to Table 2, two specific useful example glucosamine compositions are set forth.

TABLE 2
Specific embodiments of GLCN compositions.

| Composition | *Embodiment 1 | | *Embodiment 2 | |
|---|---|---|---|---|
| Component | (GP-11) | | (GP-17C) | |
| Ash Content | 0.03% | | 0.02% | |
| Si | 140 | ppm | 150 | ppm |
| Na | 10-100 | ppm | 10-100 | ppm |
| K | 10-100 | ppm | 10-100 | ppm |
| Ca | 10-100 | ppm | 10-100 | ppm |
| HCL | 0.16% | | 0.19% | |
| Citric Acid | 0.045% | | 0.074% | |
| Levulinic Acid | 0.39% | | 0.3% | |
| Melanoidins | 0.04-0.07% | | 0.02-0.03% | |
| Water-insoluble matter | 0.05% | | 0.02% | |
| soluble in gastric | | | | |
| juice at −40° | | | | |

*Percentages listed are percents by weight

The above GLCN derived from fungal biomass can be made by acid hydrolysis which breaks ether linkages in the biomass and deacetylates chitin molecules to generate free glucosamine. Acid hydrolysis can break the chitin into glucosamine, but leaves the glucosamine molecule substantially intact. Depending upon the acid hydrolysis parameters, acid hydrolysis conditions break down other components (such as glucans, proteins, and lipids) that exist in the fungal biomass.

In one specific embodiment glucosamine compositions are derived from fungal biomass by acid hydrolysis performed by treating fungal biomass for a relatively long period of time, for example greater than 4 hours, in a relatively aggressive acid solution.

Chitin-containing fungal biomass may first be reacted in a relatively aggressive acidic solution. Relatively strong (aggressive) acids may be used to hydrolyze the fungal biomass, including acids of concentrations less than 50 percent. Acids of concentrations of from 5 to 25 percent are also suitable. Suitable strong acids include hydrochloric, sulfuric, phosphoric, and citric acid at appropriate concentrations.

The aggressive acid treatment mixture containing the biomass, acid, and water is heated and maintained at a relatively elevated temperature. The mixture is usually heated to a temperature at or near its boiling point (typically 90° C. to 106° C.) and maintained under reflux conditions for 5 hours or greater, more typically greater than 8 hours, and usually less than 16 hours. The reaction may continue long enough to have a complete breakdown of the chitin, but not so long as to be inefficient or to excessively decompose the glucosamine compositions.

Although reaction in the relatively aggressive acid solution produces a glucosamine composition, subsequent purification steps may be taken. A first purification step may include a separation step, such as filtration, to remove particulate impurities, resulting in a substantially clear solution of the glucosamine composition. The solution contains a useful example glucosamine composition as well as small quantities of glucose and other components of the composition. The glucosamine composition can be concentrated and some of the acid recovered can be recycled and reused.

The glucosamine composition may be crystallized. For example, the glucosamine composition may be crystallized by adding ethanol to the concentrated solution or by continuing evaporation to the glucosamine composition solubility limit.

The glucosamine composition can be recovered by a separation process, such as filtration or centrifugation, followed by drying. The dried glucosamine composition is optionally further treated to remove undesirable residual sugars. One method of removing such sugars is by dissolving the glucosamine composition in water and adding ethanol to again precipitate the glucosamine composition while undesirable sugars remain in solution. Alternatively, the solution can be treated by electro dialysis, chromatography, membrane filtration, or other suitable procedures to further increase the concentration of glucosamine in the glucosamine composition. The glucosamine composition may optionally be decolorized and/or deodorized by, for example, treating the composition with ethanol, carbon, or other suitable material or method.

GLCN from Cartilage

In some examples, GLCN is a GLCN composition that is derived from animal cartilage (for example see U.S. Pat. No. 5,922,692). Suitable starting materials include vertebrate connective tissue, such as from a cow, pig, or chicken. Briefly, to prepare GLCN from cartilage, raw vertebrate connective tissue is disintegrated into an aggregation of particles having a substantially homogenous particle size, such as by emulsification, thereby forming liquefied connective tissue. The liquefied connective tissue is then thermally processed to generate a product rich in GLCN.

GLCN from Bacteria

In particular examples, GLCN is a GLCN composition derived from bacteria (for example see U.S. Pat. No. 6,372,457). For example, GLCN can be produced by fermentation of a microorganism. Briefly, a microorganism having a genetic modification in an amino sugar metabolic pathway is cultured in a fermentation medium. GLCN can then be recovered from the fermentation medium. Exemplary amino sugar metabolic pathways include a pathway for transport of glucosamine out of the microorganism or a pathway for transport of glucosamine into the microorganism.

NAG from Fungal Biomass

In one example, NAG is derived from fungal biomass containing chitin (for example see PCT Publication WO 03/013435). A fungal biomass that contains chitin and glucan is typically degraded to produce NAG. The chitin and glucan can be degraded enzymatically (such as using enzymes secreted by eukaryotic or prokaryotic microorganisms, for example chitinases, glucanases, and β-N-acetyl-gluosaminidases) or chemically. When enzymes are used, the degradation reaction can be maintained at a pH of from about 4.0 to about 6.0 at about 20° C. to about 45° C.

NAG from Bacteria

In certain embodiments, NAG is derived from a bacterial source (for example U.S. Patent Application No. 2002/0160459). In one embodiment, bacteria, such as E. coli, are transformed with a recombinant nucleic acid encoding N-glucosamine-6-phosphate synthase, allowing the bacteria to produce the recombinant protein, then recovering NAG from the fermentation medium.

Embodiments of the food compositions disclosed herein are further described in the following examples.

### Example 1

### Fleischmann's® Country White Bread Machine Mix

This example illustrates the sensitivity of glucosamine to baking conditions when it is incorporated into a dough and baked. Fleischmann's® Bread Machine Mix was used as the basis for incorporation of samples. Country White Mix was chosen due to its light color and mild flavor. According to the manufacturer, 1 box makes 8 servings (8 slices).

One box of bread mix was used for each batch. For servings which included GLCN or NAG, the serving (1 slice) had 0.75 g GLCN or NAG added, which reflects a typical amount of GLCN or NAG delivered in dietary supplement products. For batches with GLCN or NAG, 6 g GLCN or NAG was added (0.75 g NAG or GLCN/slice×8 slices=6 g NAG or GLCN/batch). The recipe on the box was used. GLCN or NAG was added to the dry mix and mixed with a spoon, and the mixture added to a breadmaker. To this, 8 ounces of water (75° F.-85° F.), and 1 package of yeast (provided with the bread mix) was added. The bread machine was set to medium/normal crust color. The finished bread was immediately removed, cooled on a plate, and stored in airtight containers.

Samples were tested within three days of preparing, using a “Difference From Control” test. Samples were tested blindly against a marked control, and a blind control was included. Panelists were asked to compare each sample to the control and comment. Panelists received the following instructions:
1. Taste the control sample first.
2. Compare all other samples to the control and write descriptors in the table 3. Numerically rate whether the samples are better or worse than the control, using this scale:

$${\begin{matrix}\begin{matrix}- 5 & - 4\end{matrix} & - 3\end{matrix}}_{Worse than control} \qquad - 2 \qquad - 1 \qquad {\begin{matrix}0 & + 1\end{matrix}}_{Same as control} \qquad + 2 \qquad + 3 \qquad + 4 \qquad {+ 5}_{Better than control}$$

4. Please do not discuss the results with any other panelists until all sheets are turned in.

The 10 panelists' results are shown in Table 3.

TABLE 3
Fleischmann's ® Country White Bread Machine Mix results.*

| | | | Mouthfeel/ | | |
|---|---|---|---|---|---|
| Sample | Color | Odor | texture | Taste | Comments (other than “same”) |
| Control | 0 | 0 | −0.3 | −0.2 | Less odor, drier (2) |
| NAG | −0.1 | 0 | 0 | −0.3 | Sour, darker, darker crust, tougher, heavier |
| | | | | | texture, more porous (2), salty/briny |
| | | | | | aftertaste |
| Fungal | −0.9 | −0.3 | −1.1 | −1.0 | Doughy (4), darker (2), darker crust, darker |
| GLCN | | | | | bread, denser (3), spongier, chewier (4), |
| | | | | | earthy taste, sour, sweeter (2), bitter, |
| | | | | | moister (3), “breadier” smell, crust slightly |
| | | | | | crisp, yeasty, gummy after chewed |
| Shellfish | −0.9 | −0.2 | −0.4 | −1.1 | Darker (2), darker crust, darker bread, sour, |
| GLCN | | | | | spicy/musty odor, denser, spongier (2), |
| | | | | | earthy taste, bitter taste (2), burnt taste, |
| | | | | | vinegar taste, moister, “breadier” smell, |
| | | | | | chewier, more bread taste |

*Results are the average rounded to nearest tenth.

The results shown in Table 3 are validated; based on the control score of near “0” in all categories, the control was identified.

### Example 2

### Agglomerating Material

Rice Krispies® Treats are a common snack food made by combining a crispy rice cereal with an agglomerating material that is made mainly by melting marshmallows using heat. The results provided below show that exposure of the GLCN and NAG to the heat processing conditions used to make the agglomerating material did not substantially reduce the GLCN or NAG found in the finished product. Hence, GLCN and/or NAG can be incorporated into other agglomerating materials and used with baked products.

The Rice Krispies® Treats were prepared according to the manufacturer instructions which indicate that two treats are a serving.

According to the manufacturer's instructions, the recipe prepares 24 treats. Each batch consisted of six treats, or three servings. For servings which included GLCN or NAG, the serving (2 treats) had 0.75 g GLCN or NAG, or 0.375 g per treat added, which reflects a typical amount of GLCN or NAG delivered in dietary supplement products (0.25 g-1.5 g/serving). Batches of 6 treats (3 servings) were prepared, therefore, except for the control batch, each batch had 2.25 g of GLCN or NAG added (0.375 g GLCN or NAG per treat×6 treats in batch=2.25 g GLCN or NAG per batch). GLCN or NAG was added to the melted marshmallow (melt marshmallow in a 1000 watt Amana Radarange microwave following manufacturer's instructions) prior to adding the Rice Krispies® cereal, to evenly disperse the minor dry ingredients. In this experiment, the samples were not heated to high temperatures. Results are in Table 5.

### Example 3

### Kraft Orange JELL-O®

This example provides another food portion that can be made and then layered, spread on, or otherwise combined with a baked portion to provide a food product that contains GLCN and/or NAG. Materials that can be made using similar procedures include cream fillings and puddings. These materials can then be combined with baked portions to make finished food products.

Kraft JELL-O® was used as the basis for incorporation of samples.

To make a sufficient amount of JELL-O® for sensory paneling and for recovery determinations, one-half of a box of JELL-O® was prepared for each batch. Each box of JELL-O® was divided by weight (85 g total dry mix in box was divided into two batches of 42.5 g). For servings which included GLCN or NAG, the serving had 0.75 g GLCN or NAG added, which reflects a typical amount of GLCN or NAG delivered in dietary supplement products. Because half a box is equivalent to one serving of JELL-O®, 0.75 g of GLCN or NAG was added to the dry mix of the appropriate batches. To the dry mix, 0.5 cups of boiling water were added and stirred for 2.5 minutes. Cold water (0.5 cup) was stirred in for 30 seconds, and samples refrigerated overnight.

Samples were sensory tested according to the methodology and instructions in Example 1. The 12 panelists' results are shown in Table 4.

TABLE 4
Results of JELL-O ® testing.*

| | | | Mouthfeel/ | | |
|---|---|---|---|---|---|
| Sample | Color | Odor | texture | Taste | Comments (other than “same”) |
| Control | 0 | 0 | −0.2 | −0.3 | Less tangy, sour, sweet, not as |
| | | | | | much “bite”, not as sweet, |
| | | | | | salt/sweet blend is pleasant, |
| | | | | | more fruity, texture slightly |
| | | | | | sticky |
| NAG | 0 | 0 | 0 | −0.1 | More tangy, sour, less taste, |
| | | | | | firmer texture, smoother |
| Fungal | −0.1 | 0 | −0.1 | −0.2 | Less tangy, less flavor, |
| GLCN | | | | | slightly bitter, not as sweet, |
| | | | | | firmer texture (2), slightly |
| | | | | | darker, less fruity |
| Shellfish | −0.1 | 0 | −0.3 | −0.2 | Slight aftertaste, firmer |
| GLCN | | | | | texture (2), less taste (2), not |
| | | | | | as sweet, slightly darker, a bit |
| | | | | | musky |

*Results are the average rounded to nearest tenth.

As shown in Table 3, it was difficult to ascertain differences between the samples, which shows that the exposure of GLCN and NAG to these heat processing conditions did not negatively impact the sensory traits of the finished product, and did not substantially reduce the GLCN or NAG found in the finished product (Table 5).

### Example 4

### Morsels

Chocolate morsels were melted and allowed to cool and solidify again to provide an example of a coating that can be made that includes GLCN and/or NAG. Such coating can be combined with a baked portion to make a finished product that contains GLCN and/or NAG.

Nestle Toll House® Milk Chocolate Morsels were used as the basis for incorporation of samples. One serving of morsels was estimated to be 14 g.

The shellfish GLCN is more granular than the other samples, so it was milled to disperse better in the chocolate. For servings which included GLCN or NAG, the serving (14 g) had 0.75 g GLCN or NAG added, which reflects a typical amount of GLCN or NAG delivered in dietary supplement products. As 217 g of morsels were used for the batches which had GLCN or NAG, 11.625 g of GLCN or NAG was added (217 g is 15.5 servings, 15.5 servings×0.75 g/serving=11.625 g). Each batch was heated in a 1000 watt Amana Radarange at medium-high power for one minute. The sample was stirred and again heated in the microwave for additional 20 second intervals until the chocolate was smooth. After melted the GLCN or NAG was added to the appropriate batches and stirred in quickly and thoroughly. Plastic spoons were used to drop teaspoon-sized amounts of chocolate onto wax paper, which were allowed to cool. Results are in Table 5.

### Example 5

### Cupcakes and Frosting

Cupcakes and frosting were prepared incorporating GLCN. The cupcake portion contained 18 g GLCN (24 servings×0.75 g/serving=18 g) and the control batch was prepared separately and it did not include GLCN. More specifically the following recipe was used.

Ingredients: 2⅓ cups all-purpose flour; 1½ teaspoons baking powder; ½ teaspoon baking soda; ¼ teaspoon salt; ¾ cup margarine, softened; 1⅓ cups sugar; 3 large eggs; 1 teaspoon vanilla extract; 1 cup milk mixed with 1 tablespoon distilled white vinegar; and 18 g fungal glucosamine (except for control batch).

The oven was preheated to 350° F. Paper liners were inserted into a 24 cupcake-baking pan. The following ingredients were whisked together: flour, baking powder, baking soda, and salt and GLCN (except for control batch). An electric mixer was set at medium-high speed and it was used to beat the margarine and sugar for 3 to 4 minutes until it was light and creamy. While the mixer was running the eggs where added to the butter/sugar mix. Finally, the vanilla was added to the butter, sugar and eggs. The mixer was then changed to low speed and the dry ingredients were mixed in along with the milk. The mixture was beaten until the flour was incorporated.

The dough was spooned into the cupcake liners, filling each cup about three-quarter full. The cupcakes were baked for 20 minutes at 350° F.

Weights of ingredients and final cupcakes were measured and recorded to ensure accuracy.

Frosting was prepared separately from the cupcakes, which included 9 g GLCN (12 servings×0.75 g/serving=9 g), as well as a control batch of frosting without GLCN. The recipe contained the following. Ingredients: 1 cup powdered sugar; 2 tablespoons unsalted margarine, softened; 2 tablespoons milk; ½ teaspoon vanilla extract; pinch salt; and 9 g GLCN (add GLCN to only one batch).

The frosting was prepared by mixing the margarine and powdered sugar in a bowl using an electric mixer on a medium speed. While the mixer was running 3 tablespoons of milk, the vanilla, the GLCN (except for control batch) and a pinch of salt were added. The ingredients were then beat until smooth.

The results indicted that an available GLCN diminished substantially when included in the cake portion of the cupcake and that the GLCN remained available when included in the frosting. The specific percent recover results from this example are provided in Table 5 below.

### Example 6

### Determination of the Amount of NAG Present Following Heating

To demonstrate that NAG remains following heating of food products, the following methods were used. Food samples were homogenized prior to analysis. Where possible, samples were frozen or dried and ground. Baked goods were crumbled and mixed to produce a homogeneous material. Suitable blanks (samples with no added NAG) of each food type were analyzed to assess interferences. Mass changes between unheated ingredient mixtures and baked final products were tracked to permit accurate recovery calculations.

### Acid Extraction Method

A food sample as described in the preceding examples containing 5 to 20 mg of N-acetyl-D-glucosamine (NAG) was dispersed in 25 g of 0.1 N HCl in a 50-mL polypropylene centrifuge tube and capped tightly. The sample was mixed for 30 seconds using a vortex mixer, then placed in a water bath at 37° C. The sample was removed from the water bath at 15-minute intervals and mixed for 30 seconds on a vortex mixer and then returned to the water bath. This cycle was repeated until the sample had been in the water bath for one hour. After heating, the sample was mixed for 30 seconds on a vortex mixer, then centrifuged for 10 minutes to separate the liquid and solid phases. Fats, oils or lipids in the sample formed a third layer at the top of the tube. The aqueous portion of the sample was filtered through a 0.2μ filter into an HPLC vial, then capped.

NAG recovery was determined using high performance liquid chromatography (HPLC) using a combination of refractive index and UV (195 nm) detection. The system included a SIL-10AXL autosampler, SCL-10AVP controller, LC-10AT pump, CTO-6A column oven, SPD-M10AVP diode-array detector, and a RID-6A refractive index detector, all from Shimadzu Scientific Instruments, Inc. (Columbia, Md.). The column was a MetaCarb H Plus, 300×7.8 mm, from Varian, Inc. (Torrence, Calif.).

The eluent, 0.01N sulfuric acid in water, flow rate was 0.4 mL/min. The column was maintained at 70° C. A 10 μL injection volume was used. NAG eluted at 23.9 minutes and was well resolved from other species in the samples. Multiple standards confirmed good linearity over the concentration range of interest. The UV spectrum from 190 to 350 nm indicated no measurable co-eluting peaks, and the retention time and ratio of responses between the detectors confirmed the identity of NAG.

As shown in Table 5, the amount of NAG recovered following heating ranged from about 77%-100%.

TABLE 5
Recovery of NAG and GLCN

| | | | | NAG % | GLCNHCl % |
|---|---|---|---|---|---|
| Food Product | Temp, F. | Temp, C. | Time | recovered | recovered |
| Chocolate morsels | | 52 | 1 min | 97 | 97 |
| Jello | 142 | 61 | 2.5 min | 108 | 102 |
| Bread 1 (Data from | 250 | 121 | 50 min | 100 | 46 |
| Example 1, above) | | | | | |
| Bread 2* | 250 | 121 | 50 min | Data not | 61 |
| (recipe made from scratch) | | | | collected | |
| Sugar Cookies* | 375 | 191 | 10 min | 77 | 50 |
| Corn Muffins* | 400 | 204 | 18 min | 77 | 13 |
| Cupcakes | 350 | 177 | 20 min | Data not | 56 |
| | | | | collected | |
| Cupcake Frosting | 75 | 24 | 0 | Data not | 100 |
| | | | | collected | |
| Rice Krispie binder | 165 | 74 | <10 min | Data not | 106 |
| (agglomerating material) | | | | collected | |

*Additional examples of recovery of GLCN and NAG from baked portions.

The data provided above in Table 5 demonstrates that available GLCN diminishes when it is exposed to baking conditions, and the data also shows that NAG is capable of tolerating baking conditions to a much greater extent than GLCN, but that NAG is affected under some baking conditions.

### AOAC Method

One method used to determine the amount of NAG in processed food samples was adapted from “Glucose, Fructose, Sucrose, and Maltose in Presweetened Cereals: Liquid Chromatography Method”, AOAC Method 982.14, 15^{th} Ed. (1990), pp. 789-790 (herein incorporated by reference). Specifically, section C of the method was adapted to extract NAG from dry-mixed and baked samples.

The sample was dried (if needed) then ground to render it homogeneous. Approximately five grams of sample were mixed with 100 mL of a 1:1 water:ethanol solution. The samples were heated for 30 minutes at 80-85° C. After heating, ethanol was added to replace evaporated solvent. The supernatant and solids were separated by centrifugation followed by filtration. The supernatant was analyzed by HPLC to determine the NAG content using standard methods, including a BioRad HPX-87H column heated to 60° C., 0.01 N H₂SO₄ mobile phase at 0.6 mL/minute, and a refractive index detector.

Suitable blanks (samples with no added NAG) of each food type were analyzed to assess interferences. Mass changes between dry mixes and baked final product were tracked to permit accurate recovery calculations.

Using the AOAC method NAG was recovered as follows: For bread, the dry bread mix and baked product yielded 93% and 80% recoveries of NAG, respectively. For cookies, the dry cookie mix and baked recoveries of NAG were 78% and 68%, respectively. Therefore, the majority of NAG is unchanged when exposed to a high temperature, and is available to a subject upon ingestion of the heated food product supplemented with NAG. These results compare favorably to the acid-extraction method of recovery of 100% and 77%, respectively.

### Example 8

### Determination of the Amount of GLCN Present in Processed Foods

To demonstrate that GLCN remains following heating or baking of food products, the following methods were used. Food samples were homogenized prior to analysis. Where possible, samples were frozen or dried and ground. Baked goods were crumbled and mixed to produce a homogeneous material. Suitable blanks (samples with no added GLCN) of each food type were analyzed to assess interferences. Mass changes between unheated ingredient mixtures and baked final products were tracked to permit accurate recovery calculations.

A food sample as described in the preceding examples containing 5 to 20 mg of GLCN was dispersed in 25 g of 1.0 N HCl in a 50-mL polypropylene centrifuge tube and capped tightly. The sample was mixed for 30 seconds using a vortex mixer, then placed in a water bath at 37° C. The sample was removed from the water bath at 15-minute intervals and mixed for 30 seconds on a vortex mixer and then returned to the water bath. This cycle was repeated until the sample had been in the water bath for one hour.

After heating, the sample was mixed for 30 seconds on a vortex mixer, then centrifuged for 10 minutes to separate the liquid and solid phases. Fats, oils or lipids in the sample formed a third layer at the top of the tube. A 1-g aliquot of the aqueous sample portion was diluted 100-fold with deionized water, then transferred to an autosampler vial with filter cap.

The free glucosamine in prepared samples was determined using high performance anion-exchange chromatography with pulsed amperometric detection (HPAEC-PAD). The system consisted of an EG40 eluent generator, GP50 gradient pump, AS40 autosampler, LC25 column oven, and ED40 electrochemical detector, all produced by Dionex Corporation, Sunnyvale, Calif., U.S.A.

The method was adapted from Dionex Corporation Technical Note 40. A Dionex CarboPac PA-20 column was used in place of the PA-10 described in the Technical Note. The eluent was 8 mM KOH at 0.5 mL/min. The column and detector were maintained at 30° C. The injection volume was 10 μL. The standard was glucosamine hydrochloride at 10.8 mg/L. Fermentation broth samples were diluted five-fold with deionized water, ASTM Type II, and filtered through 0.2μ vial filters in the autosampler. Multiple standards were analyzed before and after each sample set. The results are shown in Table 5 above.

In view of the many possible embodiments to which the principles of this disclosure may be applied, it should be recognized that the illustrated embodiments are only particular examples of the disclosure and should not be taken as a limitation on the scope of the disclosure. Rather, the scope of the disclosure is in accord with the following claims. We therefore claim all that comes within the scope and spirit of these claims.

1-7. (canceled)

8. A food composition comprising two components comprising:
a baked portion; and
a non-baked portion comprising a cartilage supplement selected from the group consisting of GLCN, NAG, and combinations thereof, wherein the non-baked portion is combined with the baked portion.

9. The food composition of claim 8, wherein the cartilage supplement is in a coating or glaze.

10. The food composition of claim 8, wherein the cartilage supplement is in a cereal coating.

11. The food composition of claim 8, wherein the cartilage supplement is in a cake filling.

12. The food composition of claim 8, wherein the cartilage supplement is in a pie filling.

13. The food composition of claim 8, wherein the cartilage supplement is in an agglomerating material.

14. The food composition of claim 8, wherein the cartilage supplement is in a frosting.

15. A method of making a food composition comprising:
providing at least one baked component;
providing a second component comprising a cartilage supplement selected from the group consisting of GLCN, NAG, and combinations thereof,
heat processing the second component to for a heat-processed component, wherein the heat-processed component contains at least 70% of the cartilage supplement present in the second component prior to heat processing; and
combining the at least one baked component and the at least one heat-processed component.

16. A method of making a ready-to-eat cereal comprising:
baking a first component of the cereal; and
coating the first component of the cereal with a composition comprising a cartilage supplement selected from the group consisting of GLCN, NAG or combinations thereof after baking the first component of the cereal.

17. The method of claim 16, wherein the coating is accomplished by spraying the cereal with the cartilage supplement.

18. The method of claim 16, wherein coating is accomplished by enrobing the cereal with the cartilage supplement.

19. The method of claim 16, wherein the composition comprising the cartilage supplement further comprises a sweetener.

20. A method of determining available GLCN and/or NAG in a food product comprising:
contacting the food product with a strong acid; and
separating the GLCN and/or NAG from the insoluble portion of the food product.
